(19) United States
(12) Patent Application Publication
(10) Pub. No.: US 2007/0201024 A1
(43) Pub. Date: Aug. 30, 2007

(54) METHOD AND APPARATUS FOR SEMI-PERMEABLE MEMBRANE DETECTION ON OSMOTIC TABLETS INCORATING NEAR-INFRARED SPECTROSCOPY

(76) Inventor: JOHAN H. GEERKE, Los Altos, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/679,563

(22) Filed: Feb. 27, 2007

Related U.S. Application Data
(60) Provisional application No. 60/777,993, filed on Feb. 28, 2006.

(51) Int. Cl. A61K 9/24; G01J 3/42

(52) U.S. Cl. 356/319; 424/473

(57) ABSTRACT

Disclosed are apparatus and methods relating to the manufacture of osmotic tablets, in particular to the use of near-infrared spectroscopy in the detection of the presence or absence of semi-permeable membranes in osmotic tablets.

### CROSS REFERENCE TO RELATED U.S. APPLICATION DATA

The present application is derived from and claims priority to provisional application U.S. Ser. No. 60/777,993 filed Feb. 28, 2006, which is herein incorporated by reference in its entirety.

### FIELD OF THE INVENTION

The present invention relates to the manufacture of osmotic tablets, in particular to the detection of the presence of semi-permeable membranes in osmotic tablets, and to related methods.

### BACKGROUND

Osmotic tablets in general utilize osmotic pressure to generate a driving force for imbibing fluid into a compartment formed, at least in part, by a semi-permeable membrane that permits free diffusion of fluid but not drug or osmotic agent(s), if present. A significant advantage to osmotic systems is that operation is pH-independent and thus continues at the osmotically determined rate throughout an extended time period even as the osmotic tablet transits the gastrointestinal tract and encounters differing microenvironments having significantly different pH values. A review of such osmotic tablets is found in Santus and Baker, “Osmotic drug delivery: a review of the patent literature,” Journal of Controlled Release 35 (1995) 1-21. U.S. Pat. Nos. 3,845,770; 3,916,899; 3,995,631; 4,008,719; 4,111,202; 4,160,020; 4,327,725; 4,578,075; 4,681,583; 5,019,397; and 5,156,850 disclose osmotic tablets for the continuous dispensing of active agent.

The present invention relates to osmotic tablets in which a drug composition is delivered as a slurry, suspension, erodible solid or solution at least in part by the action of an expandable (“push”) layer contained within the semi-permeable membrane. Such osmotic tablets are disclosed, among other places, in U.S. Pat. Nos. 5,633,011; 5,190,765; 5,252,338; 5,620,705; 4,931,285; 5,006,346; 5,024,842; and 5,160,743.

The presence of the semi-permeable membrane is an integral part of the functionality of osmotic tablets. Absence of the semi-permeable membrane may lead to malfunctioning of the osmotic tablet when in use. Accordingly, it is desirable to have reasonable process safeguards that check whether a semi-permeable membrane is present on osmotic tablet cores.

Accordingly, methods and apparatus are needed that provide information about whether a semi-permeable membrane is present on osmotic tablet cores, at high speeds and with low levels of errors.

### SUMMARY OF THE INVENTION

In an aspect, the invention relates to an apparatus comprising: an osmotic tablet handling system for handling osmotic tablets that comprise a semi-permeable membrane and an osmotic tablet core; an osmotic tablet removal system for removing an osmotic tablet from the osmotic tablet handling system; a near-infrared spectroscopy system; and a removal control system; wherein the osmotic tablet removal system is coupled to the osmotic tablet handling system and the near-infrared spectroscopy system, and the near-infrared spectroscopy system is coupled to the osmotic tablet handling system and a removal control system, and the removal control system is coupled to the near-infrared spectroscopy system and the osmotic tablet removal system and the osmotic tablet handling system.

In another aspect, the invention relates to a method comprising: handling an osmotic tablet that comprises a semi-permeable membrane and an osmotic tablet core; detecting differences between near-infrared spectroscopic characteristics of the semi-permeable membrane and the osmotic tablet core of the osmotic tablet being handled; and causing an osmotic tablet removal system to remove an osmotic tablet from the osmotic tablet handling system, based on the differences detected between near-infrared spectroscopic characteristics of the semi-permeable membrane and the osmotic tablet core of the osmotic tablet.

In still another aspect, the invention relates to an apparatus comprising: means for handling an osmotic tablet that comprises a semi-permeable membrane and an osmotic tablet core; means for detecting differences between near-infrared spectroscopic characteristics of the semi-permeable membrane and the osmotic tablet core of the osmotic tablet being handled; and means for causing an osmotic tablet removal system to remove an osmotic tablet from the osmotic tablet handling system, based on the differences detected between near-infrared spectroscopic characteristics of the semi-permeable membrane and the osmotic tablet core of the osmotic tablet.

### BRIEF DESCRIPTION OF THE DRAWINGS

FIG. 1 shows a schematic embodiment of the present invention with emphasis on elements of the inventive apparatus.

FIG. 2 shows infrared spectra of an osmotic tablet with and without a semi-permeable membrane being present.

FIG. 3 shows an embodiment of the present invention.

### DETAILED DESCRIPTION

I. Introduction

The inventor(s) have found unexpectedly that it is possible to solve the problems noted above with respect detecting differences between the presence and absence of a semi-permeable membrane on an osmotic tablet core of osmotic tablets by an apparatus comprising: an osmotic tablet handling system for handling osmotic tablets that comprise a semi-permeable membrane and an osmotic tablet core; an osmotic tablet removal system for removing an osmotic tablet from the osmotic tablet handling system; a near-infrared spectroscopy system; and a removal control system; wherein the osmotic tablet removal system is coupled to the osmotic tablet handling system and the near-infrared spectroscopy system, and the near-infrared spectroscopy system is coupled to the osmotic tablet handling system and a removal control system, and the removal control system is coupled to the near-infrared spectroscopy system and the osmotic tablet removal system and the osmotic tablet handling system.

Associated method embodiments of the present invention may also address the problems noted above.

As discussed further below, near-infrared spectroscopy can be used to detect differences between near-infrared spectroscopic characteristics of the semi-permeable membrane and the osmotic tablet core of an osmotic tablet. This information then can be fed to a removal control system that then can send a signal to an osmotic tablet removal system. The osmotic tablet removal system may then operate to remove an osmotic tablet from the osmotic tablet handling system. This system provides for the detection of the presence or absence of semi-permeable membranes on an osmotic tablet core of an osmotic tablet at high speeds and with low levels of error.

The invention will now be described in more detail below.

II. Definitions

All percentages are weight percent unless otherwise noted.

All references cited herein are incorporated herein by reference in their entirety and for all purposes to the same extent as if each individual publication or patent or patent application was specifically and individually indicated to be incorporated by reference in its entirety for all purposes. The discussion of references herein is intended merely to summarize the assertions made by their authors and no admission is made that any reference constitutes prior art. Applicants reserve the right to challenge the accuracy and pertinence of the cited references.

The present invention is best understood by reference to the following definitions, the drawings and exemplary disclosure provided herein.

“Drug layer” means that portion or those portions of an osmotic tablet that comprise an active pharmaceutical ingredient. Drug layers may be solid or liquid. Discussion of solid drug layers may be found in U.S. Pat. Nos. 5,633,011; 5,190,765; 5,252,338; 5,620,705; 4,931,285; 5,006,346; 5,024,842; and 5,160,743, among other places. Discussion of liquid drug layers may be found in U.S. Pat. Nos. 6,419,952; 6,174,547; 6,551,613; 5,324,280; 4,111,201; and 6,174,547, among other places.

“Near-infrared spectroscopy system” means a spectroscopy system, capable of providing near-infrared spectra of various materials, preferably osmotic tablets in an embodiment of the invention. Such near-infrared spectroscopy systems are typically coupled, preferably mechanically and/or electronically, to an osmotic tablet handling system. Such coupling facilitates the detection of differences between near-infrared spectroscopic characteristics of the semi-permeable membrane and the osmotic tablet core of an osmotic tablet being handled by the osmotic tablet handling system. In an embodiment, the near-infrared spectroscopy system is configured to detect differences between near-infrared spectroscopic characteristics of the semi-permeable membrane and the osmotic tablet core of an osmotic tablet being handled by the osmotic tablet handling system.

“Near-infrared spectroscopic characteristics of the semi-permeable membrane and the osmotic tablet core of an osmotic tablet” refer to absorbances and/or transmittances and/or reflectances, measured in the near-infrared spectrum, that are characteristic of the semi-permeable membrane and the osmotic tablet core.

“Osmotic tablet handling system” means an apparatus for handling osmotic tablets in the course of manufacturing the osmotic tablets.

“Osmotic tablets” means pharmaceutical dosage forms that are designed to operate according to osmotic principles. Examples of such osmotic tablets are provided below. In embodiments, osmotic tablets comprise a drug layer and a push layer; such embodiments are described in more detail below. Osmotic tablets according to the invention may be coated; in certain embodiments the coating(s) may be clear, translucent or opaque. In a preferred embodiment, the coatings are opaque.

“Osmotic tablet core” means the centermost portion of an osmotic tablet. The osmotic tablet core comprises the drug layer of the osmotic tablet, and in certain embodiments, also comprises the push layer.

“Osmotic tablet removal system” means an apparatus for removing and/or sorting osmotic tablets from the process stream in the course of manufacturing the osmotic tablets. Osmotic tablets that are removed from the process stream in the course of manufacturing the osmotic tablets are typically diverted to a waste stream.

“Push layer” means a displacement composition that is positioned within the osmotic tablet such that as the push layer expands during use, the materials forming the drug layer are expelled from the osmotic tablet via the at least one orifice located in the semi-permeable membrane.

“Removal control system” means a control system that operates to control the sorting actions of one or more osmotic tablet removal systems. Such removal control systems are typically coupled, preferably mechanically and/or electronically, to the near-infrared spectroscopy system and the osmotic tablet removal system. In an embodiment, the removal control system is configured to cause the osmotic tablet removal system to remove osmotic tablets from the product stream and divert them to a waste stream, based on the differences detected by the near-infrared spectroscopy system between near-infrared spectroscopic characteristics of a semi-permeable membrane and an osmotic tablet core of the osmotic tablet.

III. Osmotic Tablet Handling Systems, Osmotic Tablet Removal Systems, and Removal Control Systems

Osmotic tablet handling systems useful in the practice of this invention may be found in U.S. Pat. Nos. 5,658,474; and 5,698,119; both to Geerke. Additionally, osmotic tablet handling systems useful in the practice of this invention can be constructed from pharmaceutical tablet printing systems, such as the Delta™ series of products (available from RW Hartnett, Philadelphia, Pa.), and the VIP™ printer systems (available from Ackley Machine Corporation, Moorestown, N.J.).

Osmotic tablet removal systems may be constructed using various pharmaceutical tablet sorting systems, such as the systems available from PRODITEC (Pessac, France). Such systems are designed to remove tablets with defects from the product stream. Equivalent systems are known to one of skill in the art.

Removal control systems may be made using modified versions of removal control systems available commercially, such as those associated with the PRODITEC systems discussed above. Such systems operate to acquire defect data associated with an osmotic tablet that has been scanned by the near-infrared spectroscopy system. The removal control system then determines whether to retain the osmotic tablet in the product stream, or to divert it to waste.

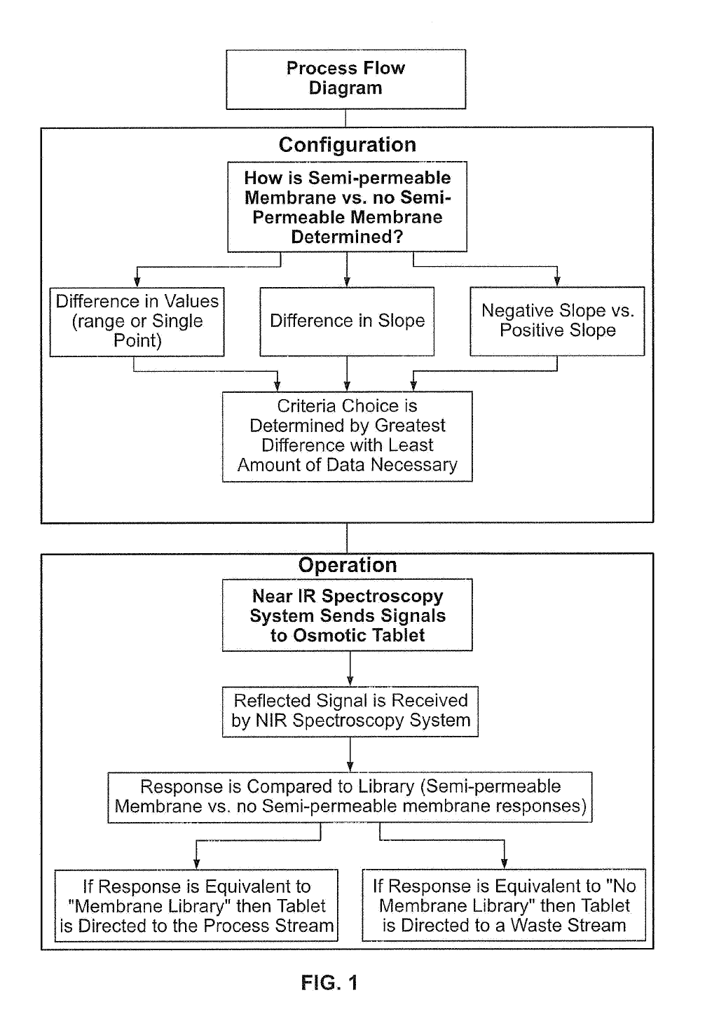

FIG. 1 shows a schematic embodiment of the present invention with emphasis on elements of the inventive apparatus. In an embodiment, data obtained from training sets of osmotic tablets is used to establish the criteria for determining the presence or absence of a semi-permeable membrane, thus achieving configuration of the apparatus. The criteria are then used to establish a library of semi-permeable membrane versus no semi-permeable membrane.

The removal control system is used to processes information generated by the near-infrared spectroscopy system that is obtained by scanning the osmotic tablet. The removal control system then controls the removal of osmotic tablets by the osmotic tablet removal system. The actual computing resources can be networked so as to be located in a convenient location. Algorithms and programs for the inventive removal control system can be conventionally modified to suit a variety of systems or components.

IV. Near Infrared Spectroscopy Systems

A variety of near-infrared spectroscopy systems are useful in the practice of this invention. Generally speaking, such systems should be capable of withstanding manufacturing environments, and capable of non-contact measurement of the osmotic tablets. Examples of equipment useful in the assembly of near-infrared spectroscopy systems include, but are not limited to, the Luminar 4030 Miniature Free Space™ Process NIR Analyzer (available from Brimrose), or the Visio Tec™ line of products (available from Uhlmann Visio-Tec GMBH, Laupheim, Germany).

Various additional equipment besides the NIR analyzer may be needed to construct a near-infrared spectroscopy system according to the invention. Such additional equipment may include, but is not limited to, air flow curtains, mounting hardware, various optical modules, computers, networking hardware, and analytical and operating software. Other hardware or software that might be needed to complete the inventive near-infrared spectroscopy system would be determinable by one of skill in the art.

In an embodiment, the near-infrared spectroscopy system comprises more than one optical module. In a preferred embodiment, the near-infrared spectroscopy system comprises at least one optical module positioned to detect the near-infrared spectroscopic characteristics of one end of the osmotic tablet, and at least one optical module positioned to detect the near-infrared spectroscopic characteristics of a different end of the osmotic tablet. The may provide the benefit of redundancy and improved reliability. Typically, the optical modules may be placed 0.5 inches or less away from the osmotic tablet being scanned, preferably 0.2 inches or less away from the osmotic tablet being scanned.

Near-infrared spectroscopy systems, and removal control systems according to the invention may be configured to detect and register differences between near-infrared spectroscopic characteristics of the osmotic tablet core and the semi-permeable membrane. Generally, the performing of the spectroscopic measurements is handled by the near-infrared spectroscopy system. The processing of those measurements to register the detection of differences may be performed wholly by the near-infrared spectroscopy system, wholly by the removal control systems, or by the combination of the two systems.

In certain preferable embodiments, the near-infrared spectroscopy system may be configured to scan the osmotic tablets in reflectance mode, although transmission mode also may be useful. An advantage of reflectance mode is that only one optical module per tablet end may be needed in certain embodiments. Preferably, the near-infrared spectroscopy system may be configured to scan wavelengths from about 1100 nm to about 2200 nm. As discussed below, once the near-infrared spectroscopic characteristics of the osmotic tablet core and the semi-permeable membrane have been determined for a particular osmotic tablet type, not all wavelengths need to be scanned during operation of the near-infrared spectroscopy system in order to detect differences between near-infrared spectroscopic characteristics of the osmotic tablet core and the semi-permeable membrane of an osmotic tablet being handled by the osmotic tablet handling system. Selecting narrower wavelength ranges provides for a faster scan speed, which may result in overall higher throughputs for the inventive apparatus or method.

There are a number of algorithms that can be used to establish differences in near-infrared spectroscopic characteristics of the osmotic tablet core and the semi-permeable membrane, once near-infrared spectra of the osmotic tablet core and the semi-permeable membrane have been determined. In a first method, if there is an obvious difference between the two near-infrared spectra (one for the osmotic tablet core and the semi-permeable membrane) then a simple difference between values at a particular wavelength, or in a different embodiment more than one wavelength, is enough to establish a differentiation.

In a second method, if there is a less than obvious difference between the two near-infrared spectra, then a region with a change in slope is chosen for evaluation. In Near-infrared Spectra #1, a region with a change in slope is chosen and an integration is performed. The difference between points on the y-axis (y2−y1) is divided by the difference between points on the x-axis (x2−x1). This will result in a slope value assigned to the Near-infrared Spectra #1. The same procedure is done for Near-infrared Spectra #2. The slope values from the two curves are compared. One value will be higher than the other. This will identify one curve with respect to the other curve. As an example, Near-infrared Spectra #1 may have a steep slope between points x2 and x1. In other words, (y2−y1) is a large number. If Near-infrared Spectra #2 is almost flat in the same region, (y2−y1) for the Near-infrared Spectra #2 will be a small number. When comparing the values of Near-infrared Spectra #1 and Near-infrared Spectra #2, one will be large and one will be small. The difference in slope values between the two near-infrared spectra can enable the detection of the osmotic tablet core versus the semi-permeable membrane.

Similarly, regions of the two near-infrared spectra can be surveyed for providing a positive slope value versus a negative slope value. This may lead to even easier identification of one near-infrared spectra versus the other, and thus to easier detection of the osmotic tablet core versus the semi-permeable membrane.

The wavelength ranges chosen for evaluation are selected after viewing several near-infrared spectra for each desired condition (membrane, no-membrane, etc). Basically a “library” is created for each desired condition. Once the region is identified, the spectrum scan is narrowed down to only look at the small region previously identified as providing a difference between the two curves. This speeds up the detection of differences between near-infrared spectroscopic characteristics of the osmotic tablet core and the semi-permeable membrane. It may also speed up the operation, because the less time it takes to establish whether the semi-permeable membrane is present or absent, the less overall time it takes to sort and otherwise process the osmotic tablet.

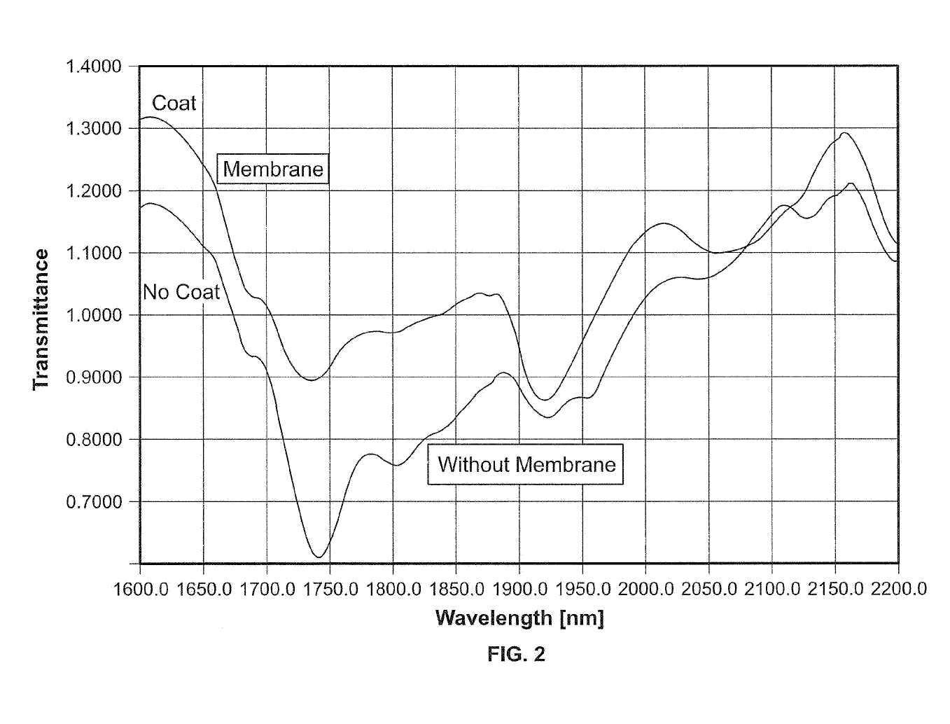

FIG. 2 shows a near-infrared spectrum obtained for an osmotic tablet with and without a semi-permeable membrane. The spectrum responses are significantly different for each condition as can be seen by inspection of FIG. 2. The difference in spectrum responses may be used, as is discussed above, to create a signal that causes the osmotic tablet removal system to remove osmotic tablets that do not comprise a semi-permeable membrane.

It is not necessary to scan the entire spectrum to make a determination. The responses at individual wavelengths or a narrow range of wavelengths may have significantly (i.e. easily detectable) different responses for the semi-permeable membrane versus the osmotic tablet core. This reduction in collection of data may result in a much more rapid process. As an example, there is a detectable different in the near-infrared spectroscopic characteristic of the osmotic tablet core and the semi-permeable membrane at about 1740 nm. In an embodiment, only this one point of the spectrum might need to be scanned to provide a reliable output.

Slope responses may also be used, as discussed above. As an example, on the “membrane” spectrum at 2120 nm, there is an upward slope, but at 2120 nm on the “without membrane” spectrum, the slope is actually negative. The difference in slopes may be enough to create a signal that causes the osmotic tablet removal system to remove osmotic tablets that lack a semi-permeable membrane.

V. Exemplary Embodiment

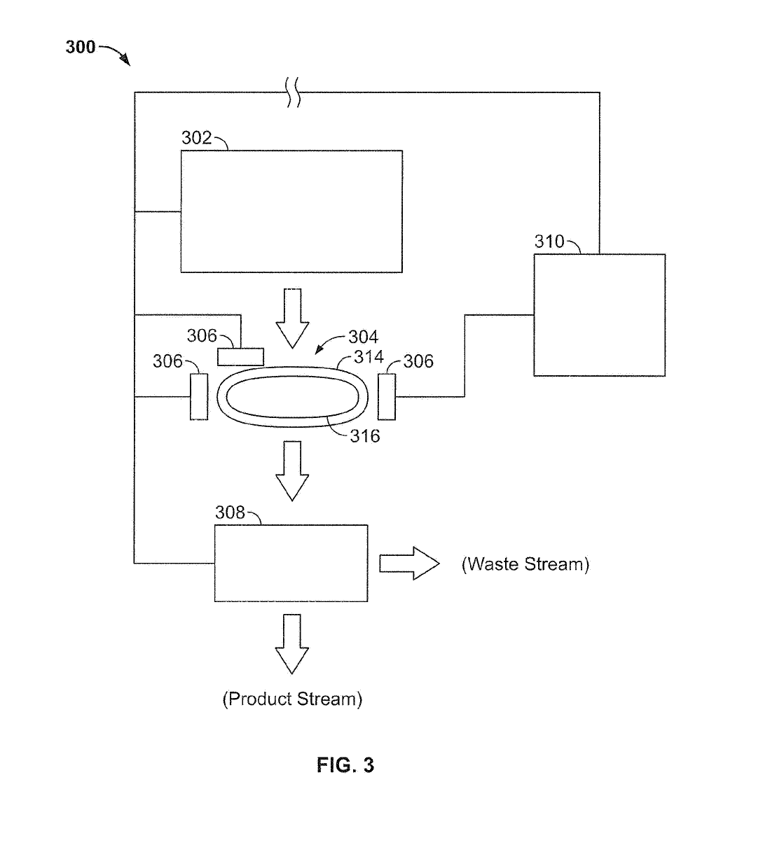

FIG. 3 shows a preferred embodiment of the present invention. Shown is embodiment 300, together with osmotic tablet handling system 302, osmotic tablet 304, near-infrared spectroscopy system 306, osmotic tablet removal system 308, removal control system 310, semi-permable membrane 314, and osmotic tablet core 316. Near-infrared spectroscopy system 306 is coupled to osmotic tablet handling system 302, and comprises three optical modules (although more or less optical modules may be used in the practice of the invention). Optional separate computer components of near-infrared spectroscopy system 306 are not shown. Osmotic tablet removal system 308 is coupled to osmotic tablet handling system 302 and removal control system 310.

In operation, osmotic tablet handling system 302 functions to place osmotic tablet 304 such that it can be scanned by near-infrared spectroscopy system 306. At that point, near-infrared spectroscopy system 306 operates as described above to detect differences between near-infrared spectroscopic characteristics of semi-permeable membrane 314 and osmotic tablet core 316. From the signal generated by near-infrared spectroscopy system 306, removal control system 310 determines the presence or absence of a semi-permeable membrane on osmotic tablet 304. Removal control system 310 then directs osmotic tablet removal system 308 to sort osmotic tablet 304 into the product stream if semi-permeable membrane 314 is detected to be present, or into the waste stream if semi-permeable membrane 314 is not detected to be present.

While there has been described and pointed out features and advantages of the invention, as applied to present embodiments, those skilled in the art will appreciate that various modifications, changes, additions, and omissions in the method described in the specification can be made without departing from the spirit of the invention. The preceding embodiments have been intended to illustrate, and in no way limit, the scope of the present invention.

What is claimed is:

1. An apparatus comprising:
an osmotic tablet handling system for handling osmotic tablets that comprise a semi-permeable membrane and an osmotic tablet core;
an osmotic tablet removal system for removing an osmotic tablet from the osmotic tablet handling system;
a near-infrared spectroscopy system; and
a removal control system;
wherein the osmotic tablet removal system is coupled to the osmotic tablet handling system and the near-infrared spectroscopy system, and the near-infrared spectroscopy system is coupled to the osmotic tablet handling system and a removal control system, and the removal control system is coupled to the near-infrared spectroscopy system and the osmotic tablet removal system and the osmotic tablet handling system.

2. The apparatus of claim 1, wherein the osmotic tablet core comprises a push layer and a drug layer.

3. The apparatus of claim 1, wherein the near-infrared spectroscopy system is configured to detect differences between near-infrared spectroscopic characteristics of the semi-permeable membrane and the osmotic tablet core.

4. The apparatus of claim 3, wherein the differences between near-infrared spectroscopic characteristics of the semi-permeable membrane and the osmotic tablet core comprise different transmittances or absorbances over at least one near-infrared wavelength.

5. The apparatus of claim 4, wherein differences between near-infrared spectroscopic characteristics of the semi-permeable membrane and the osmotic tablet core comprise different transmittances or absorbances over more than one near-infrared wavelengths.

6. The apparatus of claim 1, wherein the osmotic tablets comprise an opaque coating.

7. The apparatus of claim 1, wherein the near-infrared spectroscopy system comprises at least one optical module.

8. The apparatus of claim 7, wherein at least one optical module is positioned to detect the near-infrared spectroscopic characteristics of one end of the osmotic tablet, and at least one optical module is positioned to detect the near-infrared spectroscopic characteristics of a different end of the osmotic tablet.

9. A method comprising:
handling an osmotic tablet that comprises a semi-permeable membrane and an osmotic tablet core;
detecting differences between near-infrared spectroscopic characteristics of the semi-permeable membrane and the osmotic tablet core of the osmotic tablet being handled; and
causing an osmotic tablet removal system to remove an osmotic tablet from the osmotic tablet handling system, based on the differences detected between near-infrared spectroscopic characteristics of the semi-permeable membrane and the osmotic tablet core of the osmotic tablet.

10. The method of claim 9, wherein the osmotic tablet core comprises a push layer and a drug layer.

11. The method of claim 9, wherein the near-infrared spectroscopy system is configured to detect differences between near-infrared spectroscopic characteristics of the semi-permeable membrane and the osmotic tablet core.

12. The method of claim 11, wherein the differences between near-infrared spectroscopic characteristics of the semi-permeable membrane and the osmotic tablet core comprise different transmittances or absorbances over at least one near-infrared wavelength.

13. The method of claim 12, wherein differences between near-infrared spectroscopic characteristics of the semi-permeable membrane and the osmotic tablet core comprise different transmittances or absorbances over more than one near-infrared wavelengths.

14. The method of claim 9, wherein the osmotic tablets comprise an opaque coating.

15. The method of claim 13, wherein at least one optical module is positioned to detect the near-infrared spectroscopic characteristics of one end of the osmotic tablet, and at least one optical module is positioned to detect the near-infrared spectroscopic characteristics of a different end of the osmotic tablet.

16. An apparatus comprising:
means for handling an osmotic tablet that comprises a semi-permeable membrane and an osmotic tablet core;
means for detecting differences between near-infrared spectroscopic characteristics of the semi-permeable membrane and the osmotic tablet core of the osmotic tablet being handled; and
means for causing an osmotic tablet removal system to remove an osmotic tablet from the osmotic tablet handling system, based on the differences detected between near-infrared spectroscopic characteristics of the semi-permeable membrane and the osmotic tablet core of the osmotic tablet.

17. The apparatus of claim 15, wherein the osmotic tablet core comprises a push layer and a drug layer.

18. The apparatus of claim 15, wherein the near-infrared spectroscopy system is configured to detect differences between near-infrared spectroscopic characteristics of the semi-permeable membrane and the osmotic tablet core.

19. The apparatus of claim 17, wherein the differences between near-infrared spectroscopic characteristics of the semi-permeable membrane and the osmotic tablet core comprise different transmittances or absorbances over at least one near-infrared wavelength.

20. The apparatus of claim 18, wherein differences between near-infrared spectroscopic characteristics of the semi-permeable membrane and the osmotic tablet core comprise different transmittances or absorbances over more than one near-infrared wavelengths.

21. The apparatus of claim 15, wherein the osmotic tablets comprise an opaque coating.

22. The apparatus of claim 15, wherein the means for detecting differences between near-infrared spectroscopic characteristics of the semi-permeable membrane and the osmotic tablet core of the osmotic tablet being handled comprises a near-infrared spectroscopy system.

23. The apparatus of claim 21, wherein near-infrared spectroscopy system comprises at least one optical module.

24. The apparatus of claim 22, wherein at least one optical module is positioned to detect the near-infrared spectroscopic characteristics of one end of the osmotic tablet, and at least one optical module is positioned to detect the near-infrared spectroscopic characteristics of a different end of the osmotic tablet.
